SEQUENCE LISTING (22 sequences)

SEQ ID NO: 1 - protein, 4 aa, Escherichia coli
VMGG

SEQ ID NO: 2 - protein, 4 aa, Escherichia coli
GMGG

SEQ ID NO: 3 - protein, 4 aa, Escherichia coli
VMGG

SEQ ID NO: 4 - protein, 4 aa, Escherichia coli
GGVA

SEQ ID NO: 5 - protein, 4 aa, Escherichia coli
GRDC

SEQ ID NO: 6 - protein, 4 aa, Escherichia coli
GVGD

SEQ ID NO: 7 - protein, 4 aa, Escherichia coli
MEGD

SEQ ID NO: 8 - protein, 4 aa, Escherichia coli
GGNH

SEQ ID NO: 9 - protein, 4 aa, Escherichia coli
RESE

SEQ ID NO: 10 - protein, 4 aa, Caulobacter crescentus
RNRD

SEQ ID NO: 11 - protein, 4 aa, Caulobacter crescentus
RVDS

SEQ ID NO: 12 - protein, 4 aa, Caulobacter crescentus
RAGG

SEQ ID NO: 13 - protein, 4 aa, Caulobacter crescentus
RGQD

SEQ ID NO: 14 - DNA, 38 bp, Escherichia coli
gggatccatggctcaaggcacgctttatattgtttctg

SEQ ID NO: 15 - DNA, 28 bp, Escherichia coli
gaagcttcagtctgccaacaatttgctg

SEQ ID NO: 16 - DNA, 34 bp, Escherichia coli
gggatccatggctattgaacgtactttttccatc

SEQ ID NO: 17 - DNA, 29 bp, Escherichia coli
gaagcttaacgggtgcgcgggcacacttc

SEQ ID NO: 18 - DNA, 26 bp, Caulobacter crescentus
cagaagcgttgtcgtgcccatggttg

SEQ ID NO: 19 - DNA, 44 bp, Caulobacter crescentus
ttgcaggccaatgtggtcatgggcggcatcgtcggccgcatggg

SEQ ID NO: 20 - DNA, 32 bp, Caulobacter crescentus
ggtctagaatgaaaatctcaggcgcccggacc

SEQ ID NO: 21 - DNA, 24 bp, Caulobacter crescentus
caggatcccgatcaagcgctcctg

SEQ ID NO: 22 - DNA, 33 bp, Caulobacter crescentus
ggtctagacatatgaaaatctcaggcgcccgga

(19) United States
(12) Patent Application Publication
(10) Pub. No.: US 2011/0288286 A1
(43) Pub. Date: Nov. 24, 2011

(54) PROCESS FOR THE ENZYMATIC PRODUCTION OF CYCLIC DIGUANOSINE MONOPHOSPHATE EMPLOYING A DIGUANYLATE CYCLASE COMPRISING A MUTATED RXXD MOTIF

(76) Inventors: Thomas Simon Ilg, Zur Propstei (DE); Volker Spehr, Zur Propstei (DE); Ralf Warrass, Zur Propstei (DE)

(21) Appl. No.: 13/133,458

(22) PCT Filed: Dec. 7, 2009
(86) PCT No.: PCT/EP09/66492

§ 371 Date: Jun. 8, 2011

Related U.S. Application Data
(60) Provisional application No. 61/120,925, filed on Dec. 9, 2008.

(30) Foreign Application Priority Data
Dec. 9, 2008 (EP) 08171026.1

(51) Int. Cl. C07H 19/213; C12P 19/36; C12N 9/12

(52) U.S. Cl. 536/26.12; 435/194; 435/90

(57) ABSTRACT

A process is disclosed for the production of cyclic di-guanosine monophosphate (c-di-GMP) without the use of protecting groups by means of an enzymatic synthesis. The process comprises the coupling of two guanosine triphosphate (GTP) molecules so as to form a c-di-GMP molecule. This is done under the influence of a mutant diguanylate cyclase (DGC) comprising the amino acid sequence V153M154G155G156. It has been found that the DGC is obtainable from inclusion bodies, and therewith can be made available in amounts sufficient to improve the c-di-GMP synthesis. Particularly, the latter synthesis can be conducted in a one-pot method starting from commercially available bulk chemicals and allows upscaling to a commercial production scale.

### FIELD OF THE INVENTION

The invention is in the field of the enzymatic synthesis of cyclic di-guanosine monophosphate (c-di-GMP), by the coupling of two guanosine triphosphate (GTP) molecules under the influence of diguanylate cyclase (DGC). Particularly, the invention pertains to providing a large scale resource of DGC. Also, the invention pertains to the production of c-di-GMP on an industrial scale.

### BACKGROUND OF THE INVENTION

Cyclic di-guanosine monophosphate (c-di-GMP) is a bacterial second messenger that has been implicated in biofilm formation, antibiotic resistance, and persistence of pathogenic bacteria in their animal host.

Thus WO 2005/030186 relates to the use of c-di-GMP in a method for attenuating virulence of a microbial pathogen or for inhibiting or reducing colonization by a microbial pathogen. US 2005/0203051 relates to the use of c-di-GMP to inhibit cancer cell proliferation or to increase cancer cell apoptosis US 2006/0040887 relates to the use of c-di-GMP to stimulate or enhance immune or inflammatory response in a patient, or as an adjuvant to enhance the immune response to a vaccine. Also EP 1 782 826 relates to the use of compounds like c-di-GMP as an adjuvant for therapeutic or prophylactic vaccination, and the use thereof in a pharmaceutical composition such as a vaccine. It is further contemplated to use the compounds as active ingredients in the treatment of a wide range of infectious diseases, inflammatory diseases, autoimmune diseases, tumors, allergies, and fertility control.

For these uses, c-di-GMP is produced by means of chemical synthesis. A reference on such a synthesis is Hayakawa et al., Tetrahedron 59 (2003), 6465-6471. This document shows low yields in various synthesis steps, as is also acknowledged by the authors in a further reference, viz. Hyodo and Hayakawa, Bull. Chem. Soc. Jpn., 77, 2089-2093 (2004). However, although a considerable improvement is claimed over the very low overall yield of the previous process, it is clear that still several of the steps show great loss of product, and the overall yield is far from impressive, viz. less than 25%. The latter will be particularly hampered in view of the fact that the synthesis of c-di-GMP involves the use of a relatively high number of protecting groups throughout the molecule. The synthesis and total removal thereof presents difficulties.

It would be desired to be able to produce c-di-GMP without protecting groups e.g. by means of enzymatic synthesis. This would solve drawbacks to which the chemical synthesis is inevitably prone, such as suboptimal overall yield resulting from multistep synthesis. As M. Christen Mechanisms of c-di-GMP signaling (PhD Thesis 2007), p135 puts it: the chemical synthesis of c-di-GMP is, due to the complex synthesis of the two building blocks, of no significant commercial value.

Yet, also the enzymatic synthesis of c-di-GMP to date, however, is not suitable for production on a practical, commercial scale. It can be synthesized on a laboratory scale, yielding analytical amounts, the existence of which can be evidenced by means of HPLC, although structure identification by e.g. NMR and yields are not published. Although the technique of obtaining compounds from HPLC in itself does not preclude production on a practical, industrial scale, such as by means of preparative HPLC, the current enzymatic synthesis of c-di-GMP cannot just be scaled up.

The current enzymatic synthesis, analogously with the natural synthesis of c-di-GMP, involves the coupling of two guanosine triphosphate (GTP) molecules under the influence of diguanylate cyclase (DGC). This process is hampered by a product feedback inhibition. It is believed that an allosteric binding site for di-c-GMP is responsible for non-competitive product inhibition of DGC. In view hereof, M. Christen (2007) investigated various DGC mutants, as an aid to unravel the c-di-GMP signaling mechanisms.

DGC mutants were identified in which an RXXD motif that was found to be the core c-di-GMP binding site were changed, e.g. in the Caulobacter crescentus DgcA (CC3285) protein from R153E154S155D156 to V153M154G155G156. Herein, in accordance with the international one-letter nomenclature of amino acids, R stands for arginine, E for glutamic acid, S for serine, D for aspartic acid, V for valine, M for methionine, and G for glycine. Although therewith, by preventing the feedback inhibition, some yield improvement could be achieved, the disclosed synthesis typically leads to laboratory-scale production of milligram's, and does not allow c-di-GMP to be produced on a practical, industrial scale.

It would thus be desirable to provide an improved enzymatic synthesis of c-di-GMP, and particularly one that can be performed on a practical, industrial scale suitable for commercial production, and more particularly in a high yield and high purity.

### SUMMARY OF THE INVENTION

In order to better address one or more of the foregoing desires, the present invention, in one embodiment, presents a mutant diguanylate cyclase (DGC) comprising a modified RXXD motif, e.g. the C. crescentus DgcA (CC3285) amino acid sequence V153M154G155G156, wherein the DGC is provided in the form of inclusion bodies.

The invention, in another embodiment, presents a one-pot synthesis wherein GTP is formed by the conversion of guanosine monophosphate (GMP), comprising the addition, to a suitable reaction medium, of (a) GMP, (b) a phosphate anhydride donor, (c) a guanylate kinase (GMPK), (d) a nucleoside-diphosphate kinase (NdK), and (e) a mutant diguanylate cyclase (DGC) comprising a modified RXXD motif, e.g. the C. crescentus DgcA (CC3285) amino acid sequence V153M154G155G156, mixing, and incubating the reaction mixture, so as to form c-di-GMP.

In still another embodiment, the invention pertains to a process for the production, by enzymatic synthesis, of cyclic di-guanosine monophosphate (c-di-GMP) comprising the coupling of two guanosine triphosphate (GTP) molecules so as to form a c-di-GMP molecule, under the influence of a mutant diguanylate cyclase (DGC) comprising a modified RXXD motif, e.g. the C. crescentus DgcA (CC3285) amino acid sequence V153M154G155G156, wherein the DGC is refolded DGC obtainable from inclusion bodies.

In a further embodiment, the invention provides a method to obtain the DGC in a sufficiently high amount to conduct the foregoing reaction on a commercial scale, the method comprising the over-expression of a suitable DGC gene in a suitable host cell, and harvesting the DGC from inclusion bodies thereby obtained.

In yet a further embodiment, the invention provides a method for the isolation of pure c-di-GMP from an enzymatic reaction mixture by elution over an ion-exchange material, wherein the eluent is selected so as to obtain the c-di-GMP as the last eluted fraction.

### DETAILED DESCRIPTION OF THE INVENTION

In the following description, the term “DGC” refers to a mutant diguanylate cyclase (DGC) comprising a modified RXXD motif. For this RXXD motif on the allosteric binding site for c-di-GMP in diguanylate cyclase, reference is made to Christen et al., J. Biol. Chem., Vol. 281, Issue 42, 32015-32024, Oct. 20, 2006. The DGC preferably comprises a modified RXXD motif at amino acids 153-156, selected from the group consisting of GMGG, VMGG, GGVA, GRDC, GVGD, MEGD, GGNH, RESE, RNRD, RVDS, RAGG, and RGQD (with all letters being in accordance with the aforementioned one-letter nomenclature). Preferably the DGC is the C. crescentus mutant diguanylate cyclase DgcA (CC3285) comprising the amino acid sequence V153M154G155G156.

In a broad sense, the present invention puts to use, by substantially increasing the amount of DGC in the enzymatic coupling of GTP to c-di-GMP, a method found to obtain DGC on a relatively large scale.

According to the invention, the DGC is obtained by harvesting it as inclusion bodies upon over-expression e.g. in Escherichia coli. Inclusion bodies comprise primarily inactive, denatured protein that accumulates in intracellular aggregates. These often result from the expression of high levels of recombinant proteins in E. coli. Reference can be made to Krueger et al., “Inclusion bodies from proteins produced at high levels in Escherichia coli,” in Protein Folding, L. M. Gierasch and P. King (Eds), Am. Ass. Adv. Sci., 136-142 (1990); Marston, Biochem. J., 240:1-12 (1986); Mitraki, et al., Bio/Technol. 7: 800-807 (1989); Schein, Bio/Technol. 7:1141-1147 (1989); Taylor et al., Bio/Technol. 4: 553-557 (1986)). Inclusion bodies are dense aggregates, which are 2-3 μm in diameter and largely composed of recombinant protein, that can be separated from soluble bacterial proteins by low-speed centrifugation after cell lysis (Schoner, et al. Biotechnology 3:151-154 (1985)).

As the skilled person will understand, the DGC expressed as inclusion bodies will be denatured, and will have to be refolded into its natural conformation prior to use in the enzymatic synthesis. Techniques for the refolding of protein inclusion bodies are known to the skilled person. Isolation and purification of the refolded protein can be done in ways known in the art. The refolded protein might also be used without purification.

In a general sense, biochemists are not normally driven to produce enzymatically active proteins as inclusion bodies, particularly in view of the need to refold the protein and regain activity. In the present invention, however, the expression of DGC in inclusion bodies brings about considerable advantage for the production of c-di-GMP.

This is based, firstly, on the acknowledgement according to the invention, that the availability of DGC—which in the aforementioned Christen (2007) reference is very low—is a limiting factor for upscaling the production of c-di-GMP. In other words, the sheer amount of DGC obtainable in accordance with the invention enables a production process that can be conducted on an industrial scale.

Moreover, the very presence of DGC as inclusion bodies, rather than being traditionally disadvantageous, in the process of the invention brings about an improvement that contributes to the suitability for large scale production. Large scale expression of enzymatically active DGC protein is toxic for the protein producing cells. Hence, the DGC amounts needed for the large scale c-di-GMP production cannot reasonably be generated by the procedures of native protein expression. The present invention provides for the possibility of large-scale DgcA production as a result of the identification of a dgcA expression construct and expression conditions that allow the high level expression of enzymatically inactive inclusion bodies. The inactivity of the DgcA protein is an essential advantage in the large scale production of the enzyme, due to its toxicity to the E. coli cells. Furthermore, as the process of c-di-GMP production is dependent on DGC supply, it is important not only to produce DGC in large quantities, but also to be able to store it, and use it when needed, in the amounts needed. The harvesting of DGC as inclusion bodies provides an intrinsically stable, hence well storable, form of the protein. From this form, viz. denatured DGC, the protein can be refolded and used when desired and in the quantities as desired. DGC is typically used in an amount of at least 0.1 μM-10 μM, and preferably 1-2 μM.

The over-expression of DGC so as to produce the inclusion bodies, can be done in ways generally known in the art.

In general, recombinant constructs for the expression of target protein may be introduced into host cells using well known techniques such as electroporation and transformation. The vector may be, for example, a plasmid.

The polynucleotides encoding the target protein may be joined to a vector containing a selectable marker for propagation in a host. Preferred are vectors comprising cis-acting control regions to the polynucleotide of interest. Appropriate trans-acting factors may be supplied by the host, supplied by a complementing vector or supplied by the vector itself upon introduction into the host.

Vectors that provide for specific expression, include those that may be inducible. Particularly preferred among such vectors are those inducible by environmental factors that are easy to manipulate, such as temperature and nutrient additives.

Expression vectors useful for the expression of target proteins include e.g., vectors derived from bacterial plasmids.

The DNA insert containing the gene for the target protein should be operatively linked to an appropriate promoter, such as the phage T7. Other suitable promoters will be known to the skilled artisan. The expression constructs will further contain sites for transcription initiation, termination and, in the transcribed region, a ribosome binding site for translation. The coding portion of the target transcripts expressed by the constructs will preferably include a translation initiating at the beginning and a termination codon (UAA, UGA or UAG) appropriately positioned at the end of the polypeptide to be translated.

As indicated, the expression vectors will preferably include at least one selectable marker. Such markers include tetracycline, kanamycin, chloramphenicol or ampicillin resistance genes for culturing in E. coli and other bacteria. Representative examples of appropriate hosts include, but are not limited to, bacterial cells, such as E. coli. Appropriate culture mediums and conditions for the above-described host cells are known in the art.

Among vectors preferred for use in bacteria expression of target proteins include pET vectors, available from Novagen. Other suitable vectors will be readily apparent to the skilled artisan.

In a particularly preferred embodiment of the invention, the c-di-GMP is produced in a one-pot reaction. Herein GTP is formed by the conversion of guanosine monophosphate (GMP), comprising the addition, to a suitable reaction medium, of (a) GMP, (b) a phosphate anhydride donor, (c) a guanylate kinase (GMPK), (d) a nucleoside-diphosphate kinase (NdK), and (e) the DGC, mixing, and incubating so as to form c-di-GMP.

It will be apparent to the skilled person that a one-pot synthesis is greatly preferred. In general, a one-pot synthesis avoids loss of yield normally incurred when isolating and purifying reaction intermediates. Moreover, in the present case the possibility to start from GMP rather than GTP brings about a further benefit for commercial production, since GTP is scarce and expensive, and therewith forms another limiting factor for industrial upscaling.

The one-pot synthesis is depicted in the following scheme.

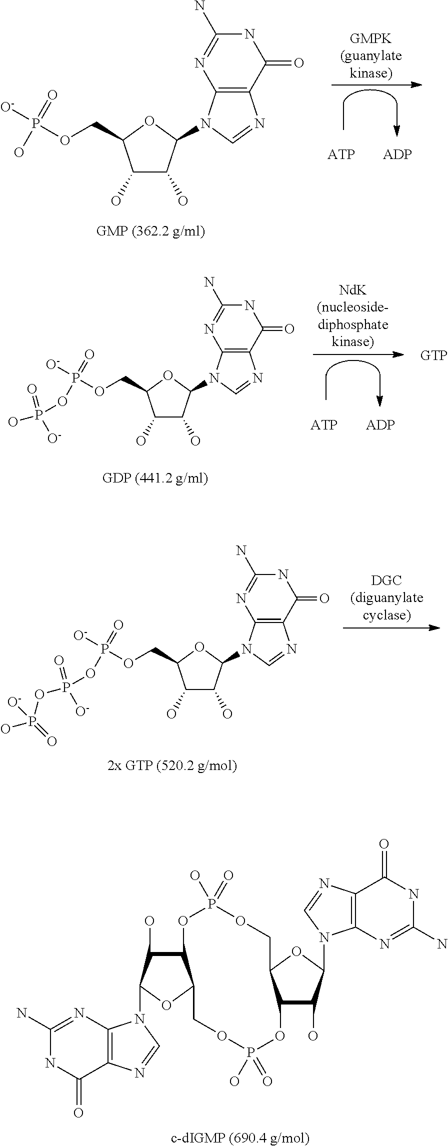

Herein ATP is adenosine triphosphate, ADP is adenosine diphosphate, and the other abbreviations have been given above.

The availability of DGC is key to the one-pot process, as the sequence of steps up to the formation of GTP is reversible. Hence, with all reactants present in a single reaction medium, it is imperative that GTP be removed so as to drive the reaction towards the formation of c-di-GMP. The present invention makes it possible to remove GTP by its conversion (i.e. coupling) into c-di-GMP. The availability of the aforementioned large amount of DGC is the key tool for this. This allows supplementing DGC during synthesis, i.e. providing a constant supply of DGC so as to drive the reaction in which GTP is coupled to form c-di-GMP, and is removed.

The conditions for the one-pot synthesis can be well determined by the skilled person. Suitable reaction media are e.g. aqueous buffers of slightly alkaline pH comprising Tris (tris(hydroxymethyl)aminomethane) as a buffering agent. Other buffering agents are known. Typical amounts of reactants and enzymes are in the range of 0.01-1 U/ml for NdK and GMPK and 0.1-10 μM for DgcA; as reactants, 0.1-20 mM GMP and 0.2-50 mM ATP. Suitable phosphate anhydride donors are known, with the best-known and preferred example being ATP (adenosine triphosphate). Any NdK and GMPK enzymes from different species can be used although the E. coli enzymes being the best characterized.

Apart from the DGC, which can be obtained in accordance with the present invention, the starting materials used in the enzymatic synthesis are commercially available or can be prepared and isolated in manners known per se in the art.

The isolation and purification of c-di-GMP obtained in accordance with the invention can be done in ways generally known in the art. These generally include elution over ion exchange materials, typically ion exchange columns. In this respect the invention provides a further advantageous embodiment. Surprisingly, the mixture resulting from the enzymatic one-pot process described hereinbefore, allows elution (by judicious choice of eluent) of all other components prior to elution of c-di-GMP. A typical eluent is low concentrated HCl with lithium chloride. All reagents and by-products can be separated from c-di-GMP by washing with water, ammonium acetate, 20 mM HCl and 40 mM LiCl containing aqueous solutions. Product is finally eluted from the column in highly concentrated fractions by 20 mM HCl/500 mM LiCl. Final purification is done by precipitation of the aqueous solution of c-di-GMP in acetone:EtOH or in other organic/water miscible solvent systems. The addition of ammonia provides the di ammonium salt of c-di-GMP.

Although the one-pot enzymatic synthesis has been made possible solely as a result of the availability of DGC in large amounts, as inclusion bodies, it will be understood that the advantages associated with the one-pot synthesis itself, could also be enjoyed if sufficient DGC were available from another source.

Preferably, the DGC used in the one-pot synthesis is in fact refolded DGC obtainable from inclusion bodies as described hereinbefore.

The enzymatic synthesis of c-di-GMP of the invention essentially enables production on a commercial, industrial scale. The terms “commercial scale” and “industrial scale” are employed to distinguish the production scale from that typically found in a laboratory (described in the before mentioned publications). The latter generally involves a scale of production of not far above 10 mg at most. Commercial scale will involve tens to hundreds of grams, up to kilogram's scale. In the present invention production is on a scale of at least 1 gram, particularly a scale of tens of grams, preferably at least hundred grams, and more preferably at least one kg.

The upscaling itself, now that the limiting availability of DGC has been resolved in accordance with the invention, can be done in ways standard in the art.

The invention herewith also pertains to an article of manufacture in the form of a batch of at least 10 g, preferably at least 100 g and more preferably at least 1 kg, of c-di-GMP of over 95% purity (according to HPLC analysis), and particularly of 100% purity (according to HPLC analysis), obtainable by enzymatic synthesis according to the methods described hereinbefore. Quantitative analysis of c-di-GMP (synthesized by the method described above) by NMR analysis reveal a purity in the range of 80-90% (w/w). The mass balance can be completed by the addition of ammonia (typically in the range of 5% (w/w)), water (typically in the range of 5-15% (w/w)) and residual amounts of anionic and kationic salts (e.g. Li⁺, Na₊, PO₄²⁻, Cl⁻).

The c-di-GMP of the invention can be put to use in its normal way, yet with the benefit of commercial scale production, combined with the high purity associated with enzymatic synthesis. The large amounts of c-di-GMP available by the present invention make the use of c-di-GMP in the treatment of infections and other diseases in human and animal health possible. Large scale synthesis make also the semi-synthetic production of c-di-GMP derivatives possible, e.g. the production of thio-phosphates, phosphate esters, acetylated and alkylated c-di-GMP derivatives become accessible by the chemical transformation of c-ci-GMP.

The invention will be further explained hereinafter with reference to the following non-limiting Examples, and the accompanying Figures.

### Example 1

This Example describes the gene cloning, overexpression, purification and characterization of guanylate kinase and nucleoside diphosphate kinase from Escherichia coli
a) Gene Cloning of E. coli gmpk and E. coli ndk
Based on the Genbank database DNA sequences of E. coli GmpK (M84400) and E. coli NdK (X57555), primer were designed for PCR amplification of the respective genes:

Ec-gmpK-for
GGGATCCATGGCTCAAGGCACGCTTTATATTGTTTCTG
Ec-gmpK-rev
GAAGCTTCAGTCTGCCAACAATTTGCTG
Ec-ndk-for
GGGATCCATGGCTATTGAACGTACTTTTTCCATC
Ec-ndk-rev
GAAGCTTAACGGGTGCGCGGGCACACTTC

Introduced cloning sites are underlined. Start and stop codons of the genes are in bold.
Genomic DNA was isolated from E. coli JM109 by standard methods (Joseph Sambrook and David W. Russell. Molecular Cloning: A Laboratory Manual. Cold Spring Harbor Laboratory Press, Cold Spring Harbor, N.Y.). PCR was performed using 4 ng/ml genomic DNA template and 0.5 M of each primer in standard PCRs (30 cycles, 30 sec extension time, 57° C. and 62° C., respectively, annealing temperature). The expected DNA bands representing the gmpK and ndK genes were observed after 1% TAE agarose gel electrophoresis.
The respective PCR bands were excised, the DNA fragments purified by GeneClean^{R}, and ligated into pCR2.1-Topo. Two independent plasmid clones were isolated for GmpK and NdK, respectively, and the DNA inserts were sequenced. The deduced protein sequence of all plasmid clone inserts were identical to the respective database protein sequences.
b) GmpK and NdK Overexpression Experiments in E. coli
For overexpression experiments, the open reading frames of E. coli gmpk and E. coli ndk were subcloned via the introduced flanking BamHI and HindIII sites into BamHI/HindIII-cut pQE30.
The resulting plasmids were then introduced into E. coli M15. Expression experiments were performed by standard protocols (briefly: dilution of overnight LB-Amp-Kan cultures 1+9 in fresh LB-Amp-Kan, growth for 2 h at 37° C., addition of 1 mM IPTG, growth for a further 4.5 h at 37° C., harvest).

### Procedure for the Purification of Both GmpK and NdK:

The harvested cell pellets of 1 liter IPTG-induced culture had been frozen at −20° C. until further processing: the frozen pellets were thoroughly resuspended in 40 ml lysis buffer (50 mM NaH₂PO₄, 300 mM NaCl, 10 mM imidazole, pH 8.0/NaOH) supplemented with: 0.5% Triton X100, 1 mg/ml lysozyme, 25 U/ml benzonuclease. The suspensions were incubated on ice for 1 mg/ml h ( [symbol] lysate; Lys) followed by a 1 h centrifugation at 2500 g. The pellet was washed once with 10 ml lysis buffer with supplements and centrifuged. The washed pellet was resuspended in 50 ml 2% SDS for SDS polyacrylamide gel electrophoresis (SDS-PAGE) analysis ( [symbol] Pellet; Pe). The combined centrifugation supernatants (Sn) were then applied onto a 4 ml Ni²⁺-NTA agarose (Qiagen) column, that was preequilibrated with lysis buffer without supplements. The flowthrough was collected (Ft) and the column was washed with 5 column volumes wash buffer (50 mM NaH₂PO₄, 300 mM NaCl, 20 mM imidazole, pH 8.0/NaOH). Elution was performed with elution buffer (50 mM NaH₂PO₄, 300 mM NaCl, 250 mM imidazole, pH 8.0/NaOH), and 5 ml fractions were collected (Elu 1-5). All fractions of the lysate processing and the Ni²⁺-NTA agarose column eluate were analysed by 12% SDS-PAGE and Coomassie blue staining.

### Example 2

### a) Gene Cloning of C. Crescentus dgcA

Based on the Genbank database DNA sequences of C. crescentus dgcA (cc_{—}3285; ACCESSION AE005673) primer were designed for PCR amplification of the respective genes:

Cacr-002
CAGAAGCGTTGTCGTGCCCATGGTTG
Cacr-004
TTGCAGGCCAATGTGGTCATGGGCGGCATCGTCGGCCGCATGGG
Cacr-010
GGTCTAGAATGAAAATCTCAGGCGCCCGGACC
Cacr-011
CAGGATCCCGATCAAGCGCTCCTG
Cacr-014
GGTCTAGACATATGAAAATCTCAGGCGCCCGGA

Introduced cloning sites are underlined. Start codon of dgcA is in bold.
PCR was performed using 4 ng/ml genomic DNA of C. crescentus CB15 template and 1.0 μM of primer Cacr-002/Cacr-004 in a standard PCR (35 cycles, 30 sec extension time, 55° C., annealing temperature). The expected DNA bands representing the 3′-end of the dgcA gene including the R153V-E154M-S155G-D155G mutation was observed after 1% TBE agarose gel electrophoresis. The respective PCR band was excised, the DNA fragment purified by QIAquick PCR Purification Kit (Qiagen) and used as a megaprimer in the next PCR reaction in combination with primer 1.0 μM Cacr-010 primer and 4 ng/ml genomic DNA of C. crescentus CB15 template. The expected DNA bands representing the complete dgcA gene including the R153V-E154M-S155G-D155G mutation was observed after 1% TBE agarose gel electrophoresis. The respective PCR bands were excised, the DNA fragments purified by QIAquick PCR Purification Kit (Qiagen) and ligated into pCR-II TOPO to form pCacr-003b. Two independent plasmid clones were isolated for DgcA_{VMGG} and the DNA inserts were sequenced. The deduced protein sequence of all plasmid clone inserts were identical to the respective database protein sequences, instead of carrying the R153V-E154M-S155G-D155G mutation.
Another PCR was performed using 1 ng/ml pCacr-003b template and 1.0 μM of primer Cacr-011/Cacr-014 in a standard PCR (35 cycles, 30 sec extension time, 55° C., annealing temperature). The expected DNA band representing the dgcA_{VMGG} gene including the R153V-E154M-S155G-D155G mutation was observed after 1% TBE agarose gel electrophoresis. The respective PCR band was excised, the DNA fragment purified by QIAquick PCR Purification Kit (Qiagen) and ligated into pCR-II TOPO to form pCacr-018a. Two independent plasmid clones were isolated for DgcA_{VMGG} and the DNA inserts were sequenced. The deduced protein sequence of all plasmid clone inserts were identical to the respective database protein sequences, instead of carrying the R153V-E154M-S155G-D155G mutation.

### b) This Examples Illustrates Obtaining DgcA from C. Crescentus

For overexpression experiments, the open reading frames of C. crescentus dgcA_{VMGG} from plasmid pCacr-18a was subcloned via the flanking BamHI and NdeI sites into BamHI/NdeI-cut pET-15b. The resulting plasmid pCacr-20 was then introduced into E. coli BL21(DE3).
E. coli BL21(DE3) cells carrying the expression plasmid pCacr-20 were grown in LB medium with ampicillin (100 μg/ml) at 37° C., and expression was induced by adding isopropyl 1-thio-β-D-galactopyranoside at A₆₀₀ 0.4 to a final concentration of 1 mM. After induction cells were grown for additional 4 h at 37° C. After harvesting by centrifugation, cells were resuspended in buffer containing 50 mM Tris-HCl, pH 8.0, 50 mM NaCl, 0.5 mM EDTA, 5% Glycerol, 20 μl/g cell pellet Lysonase (Novagen), incubated for 15 min at room temperature, and lysed by passage through a French pressure cell. 3-(1-Pyridino)-1-propane sulfonate (NDSB-201) was added to a final concentration of 125 mM, and the mixture was incubated for another 15 min at room temperature. Soluble and insoluble protein fractions were separated by centrifugation for 15 min at 8,000×g. The pellet containing the inclusion bodies was washed once with wash buffer (10 ml/g cell pellet) containing 50 mM Tris-HCl, pH 8.0, 50 mM NaCl, 0.5 mM EDTA, 5% Glycerol, 1 mM tris(2-carboxyethyl)phosphine (TCEP), and 125 mM NDSB-201 and centrifugated for 15 min at 8,000×g. The inclusion bodies were washed twice with resuspension buffer (10 ml/g cell pellet) containing 50 mM Tris-HCl, pH 8.0, 50 mM NaCl, 0.5 mM EDTA, 5% Glycerol, and 1 mM TCEP and centrifugated for 15 min at 8,000×g. The purified inclusion bodies were stored at −80° C. or resuspended in buffer containing 50 mM Tris-HCl, pH 8.0, 200 mM NaCl, 2 mM EDTA, 7 M guanidine hydrochloride and 10 mM TCEP by stirring for 60 min at room temperature. Soluble and insoluble protein fractions were separated by centrifugation for 15 min at 25,000×g and 4° C. The supernatant containing the denaturated DgcA was sterilized by filtration through a 0.45 μm filter and stored at −80° C. until use. For refolding, denaturated DgcA (5 mg/ml) was added to 25 vol. buffer containing 500 mM L-Arginine, 50 mM HEPES, pH 7.5 and incubated with stirring at 4° C. for 18 h.

### Example 3

This Examples illustrates the production of c-di-GMP from GMP 1250 ml freshly refolded DgcA, 5000 U NdK (2.8 U/μl in 50% Glycerol), 5000 U GmpK (3.6 U/μl in 50% Glycerol), Guanosine 5′-monophosphoric acid (GMP, 4 g, 11 mmol), 13.75 g Adenosine 5′-triphosphoric acid disodium salt (ATP), and 3750 ml reaction buffer containing 50 mM Tris-HCl, pH 7.5, 10 mM MgCl₂, 0.5 mM EDTA and 50 mM NaCl were mixed and incubated, slightly shaking with 80 rpm, at 30° C. for 16 h.

### Purification

The crude reaction mixture (4800 ml) was filtered through cellulose and added to an anion exchange column (Dowex 1×2, 400 ml Pharmacia XK26, 2.5 cm inner diameter, approx. 70 cm length; Cl-form, extensively washed with 0.5% acetic acid, equilibrated with water (2000 ml); flow rate 10 ml/min).

The column was washed with water (2000 ml, 10 ml/min), 2M NH₄OAc (2000 ml, 10 ml/min), water (1500 ml, 10 ml/min), 10 mM HCl/50 mM LiCl (1000 ml, 10 ml/min).

Product was eluted from the ion exchange column by 10 mM HCl/500 mM LiCl (2000 ml, 10 ml/min).

Aqueous eluent of an ion-exchange chromatography containing product (approx. 800 ml) was adjusted to basic pH by the addition of ammonia (1.5 ml, 32% w/w). The solvent was evaporated under reduced pressure resulting in a highly viscose suspension. Product was precipitated by the addition of EtOH:aceton (1;1 (v:v); 300 ml) and stirred for 15 min at room temperature. The solid was separated by filtration (pore 3), dissolved in 5% NH₃ (25 ml) and was again precipitated by the addition of EtOH:Aceton (1;1; (v:v); 300 ml). The dissolution/precipitation procedure was repeated once. The obtained solid was dissolved in 1% NH₃ (20 ml), filtered (0.45 μm pore size; PET filter) and lyophilized over night.

Yield c-di-GMP×2 NH₃ (1.79 g, 2.5 mmol) was obtained as an off-white solid in 45% overall yield (calculation based on starting material GMP).

### Product Characterization

The identity of enzymatically produced c-di-GMP was confirmed by NMR and LCMS by comparing data to chemically synthesized c-di-GMP. No impurities were detected by LCMS and NMR.

¹H-NMR: s (8.0, 2H), s (6.0, 2H), m (5.0, 2H), m (4.8, 2H+H₂O signal), m (4.4, 4H), m (4.1, 2H); HPLC-MS: 3.36 min (purity 100% at 210 and 254 nm); [M+1]=691 (th. 691). HPLC: Atlantis-HPLC-Column, 4.6*50 mm, dC18, 3 μm; Solvent system: Water (+0.1% formic acid)=solvent A; Acetonitril (+0.1% formic acid)=solvent B; method: 0-10% B (=100-90% A) in 5 min; 1 min at 10% B; total runtime: 8 min. Quantitative NMR analysis determined high c-di-GMP purity (w/w %=82.1%). Ion chromatography determined the presence of sodium (w/w %=0.4%), ammonium (w/w %=5.3%) and lithium (w/w %=0.04%) cations and small amounts of phosphate (w/w %=0.15%) and chloride anions (w/w %=0.7%) in the final product. Karl-Fischer titration was used to determine the amount of water. The measurement was performed in MeOH suspension (due to the insolubility of the c-di-GMP in non-aqueous solvent systems). The determined amount of water (w/w %=approx. 10%) is used as a rough estimation.

1. A mutant diguanylate cyclase (DGC) comprising a modified RXXD motif, wherein the DGC is provided in the form of inclusion bodies.

2. A mutant DGC according to claim 1, comprising a modified RXXD motif at amino acids 153-156, selected from the group consisting of GMGG (SEQ ID NO.: 2), VMGG (SEQ ID NO.: 1), GGVA (SEQ ID NO.: 4), GRDC (SEQ ID NO.: 5), GVGD (SEQ ID NO.: 6), MEGD (SEQ ID NO.: 7), GGNH (SEQ ID NO.: 8), RESE (SEQ ID NO.: 9), RNRD (SEQ ID NO.: 10), RVDS (SEQ ID NO.: 11), RAGG (SEQ ID NO.: 12), and RGQD (SEQ ID NO.: 13).

3. A mutant DGC according to claim 2, having the C. crescentus DgcA (CC3285) amino acid sequence V153M154G155G156.

4. A method for the manufacture of recombinant DGC according to claim 1, comprising the over-expression of a suitable DGC gene in a suitable host cell, and harvesting the DGC from inclusion bodies thereby obtained.

5. A process for the production, by enzymatic synthesis, of cyclic di-guanosine monophosphate (c-di-GMP) comprising the coupling of two guanosine triphosphate (GTP) molecules so as to form a c-di-GMP molecule, under the influence of a mutant diguanylate cyclase (DGC) comprising a modified RXXD motif, conducted in a one-pot reaction wherein GTP is formed by the conversion of guanosine monophosphate (GMP), comprising the addition, to a suitable reaction medium, of (a) GMP, (b) a phosphate anhydride donor, (c) a guanylate kinase (GmpK), (d) a nucleoside-diphosphate kinase (NdK), and (e) the DGC, mixing, and incubating the reaction mixture, so as to form c-di-GMP.

6. A process according to claim 5, wherein the DGC is refolded DGC obtainable from inclusion bodies.

7. A process according to claim 5, wherein the DGC is used in an amount of 0.1 μM-10 μM.

8. A process according to claim 7, wherein the DGC is used in an amount of 1-2 μM.

9. A process for the production, by enzymatic synthesis, of cyclic di-guanosine monophosphate (c-di-GMP) comprising the coupling of two guanosine triphosphate (GTP) molecules so as to form a c-di-GMP molecule, under the influence of a mutant diguanylate cyclase (DGC) comprising a modified RXXD motif, wherein the DGC is provided in the form of inclusion bodies.

10. A process according to claim 5, wherein the DGC is supplemented during the synthesis.

11. A batch of at least 10 g of c-di-GMP of over 99% purity, obtainable by a process according to claim 5.

12. A batch according to claim 11, having a size of at least 100 g.

13. The process of claim 6, wherein the mutant DGC comprises a modified RXXD motif at amino acids 153-156, selected from the group consisting of GMGG (SEQ ID NO.: 2), VMGG (SEQ ID NO.: 1), GGVA (SEQ ID NO.: 4), GRDC (SEQ ID NO.: 5), GVGD (SEQ ID NO.: 6), MEGD (SEQ ID NO.: 7), GGNH (SEQ ID NO.: 8), RESE (SEQ ID NO.: 9), RNRD (SEQ ID NO.10), RVDS (SEQ ID NO.: 11), RAGG (SEQ ID NO.: 12), and RGQD (SEQ ID NO.: 13).

14. The process of claim 6, wherein the mutant DGC has the C. crescentus DgcA (CC3285) amino acid sequence V153M154G155G156.

15. The process of claim 9, wherein the mutant DGC comprises a modified RXXD motif at amino acids 153-156, selected from the group consisting of GMGG (SEQ ID NO.: 2), VMGG (SEQ ID NO.: 1), GGVA (SEQ ID NO.: 4), GRDC (SEQ ID NO.: 5), GVGD (SEQ ID NO.: 6), MEGD (SEQ ID NO.: 7), GGNH (SEQ ID NO.: 8), RESE (SEQ ID NO.: 9), RNRD (SEQ ID NO.10), RVDS (SEQ ID NO.: 11), RAGG (SEQ ID NO.: 12), and RGQD (SEQ ID NO.: 13).

16. The process of claim 9, wherein the mutant DGC has the C. crescentus DgcA (CC3285) amino acid sequence V153M154G155G156.

17. A method for the manufacture of recombinant DGC according to claim 2, comprising the over-expression of a suitable DGC gene in a suitable host cell, and harvesting the DGC from inclusion bodies thereby obtained.

18. A method for the manufacture of recombinant DGC according to claim 3, comprising the over-expression of a suitable DGC gene in a suitable host cell, and harvesting the DGC from inclusion bodies thereby obtained.
